(19) United States
(12) Patent Application Publication
(10) Pub. No.: US 2019/0076120 A1
(43) Pub. Date: Mar. 14, 2019

(54) SYSTEMS AND METHODS FOR GUIDING ULTRASOUND PROBE PLACEMENT USING HAPTIC FEEDBACK

(71) Applicant: General Electric Company, Schenectady, NY (US)

(72) Inventor: David Andrew Shoudy, Niskayuna, NY (US)

(21) Appl. No.: 16/110,923

(22) Filed: Aug. 23, 2018

Related U.S. Application Data
(60) Provisional application No. 62/556,169, filed on Sep. 8, 2017.

(51) Int. Cl. A61B 8/00; A61B 34/00

(52) U.S. Cl. CPC A61B 8/4444; A61B 8/461; A61B 34/76; A61B 8/4263; A61B 8/4254; A61B 8/54

(57) ABSTRACT

In accordance with the present approach, an operator may receive assistance in positioning and/or orienting an ultrasound probe so as to acquire a target scan plane of a target anatomy. In accordance with certain embodiments, an ultrasound imaging system and ultrasound probe may be provided that provide haptic feedback guiding ultrasound probe placement. Such automation and guidance may allow novice, or less skilled, users to obtain successful ultrasound scans.

### CROSS-REFERENCE TO RELATED APPLICATIONS

This application claims priority to U.S. Patent Provisional Application No. 62/556,169, entitled “SYSTEMS AND METHODS FOR GUIDING ULTRASOUND PROBE PLACEMENT USING HAPTIC FEEDBACK”, filed Sep. 8, 2017, which is herein incorporated by reference in its entirety.

### BACKGROUND

The subject matter disclosed herein relates to medical imaging, and more particularly to systems and methods for guiding ultrasound probe positioning through haptic feedback.

An ultrasound imaging system typically includes an ultrasound probe that is applied to a patient's body and a workstation or monitor that is operably coupled to the ultrasound probe. The ultrasound probe may be controlled by an operator of the ultrasound imaging system and is configured to transmit and receive ultrasound signals that are processed into an ultrasound image by the workstation or monitor. The operator positions the ultrasound probe to acquire a target anatomy or region of interest (e.g., a desired tissue or body region to be imaged) in a target scan plane. For example, by viewing real-time images of the acquired ultrasound data on the monitor or a separate display of the ultrasound imaging system, the operator may adjust the ultrasound probe into an appropriate position for imaging the target scan plane of the target region of interest. However, it is recognized that there may be some challenges with such positioning methods. For example, manually finding the appropriate position of the ultrasound probe via viewing the displayed images alone may be difficult, time consuming, and result in less accurate positioning, especially for unskilled users.

### BRIEF DESCRIPTION

In one embodiment, an ultrasound imaging system includes an ultrasound probe having a handle with a plurality of haptic actuators. The ultrasound imaging system further includes a monitor having a memory and a processor. The processor is communicatively coupled to the ultrasound probe and is configured to determine a current position of the ultrasound probe relative to the target region of interest, determine whether the current position of the ultrasound probe corresponds to a target position of the ultrasound probe relative to a target region of interest of a patient, wherein the target region of interest comprises a target anatomy and a target scan plane, and output, in response to determining that the current position of the ultrasound probe relative to the target region of interest does not correspond to the target position of the ultrasound probe, a control signal to one or more of the haptic actuators of the plurality of haptic actuators. The control signal causes one or more of the haptic actuators to activate in an activation pattern indicative of a suggested movement of the ultrasound probe to position the ultrasound probe in the target position.

In another embodiment, an ultrasound probe includes a patient-facing surface having one or more transducers. The ultrasound probe further includes a handle having a plurality of haptic actuators. The haptic actuators are configured to actuate in a plurality of actuation patterns to indicate a suggested movement of the handle in six degrees-of-freedom. Each actuation pattern of the plurality of actuation patterns modulates in intensity, speed, or both as the patient-facing surface of the ultrasound probe is moved closer to a target position relative to a patient.

In another embodiment, a method, includes receiving, via a processor, a target region of interest of a patient to be imaged via an ultrasound probe of an ultrasound imaging system and a target scan plane of the target region of interest, and determining, via the processor, a target imaging position of the ultrasound probe based at least in part on the target region of interest and the target scan plane. The method further includes determining, via the processor, a current position of the ultrasound probe relative to the target region of interest, the desired scan plane, or both, and determining, via the processor, whether the current position of the ultrasound probe corresponds to the target imaging position of the ultrasound probe. The method further includes outputting, via the processor, in response to determining that the current position of the ultrasound probe relative to the target region of interest, the target scan plane, or both, does not correspond to the target position of the ultrasound probe, a control signal to a plurality of haptic actuators disposed on a handle of the ultrasound probe. The control signal causes one or more of the haptic actuators to actuate in an actuation pattern indicative of a suggested movement of the ultrasound probe.

### BRIEF DESCRIPTION OF THE DRAWINGS

These and other features, aspects, and advantages of the present disclosure will become better understood when the following detailed description is read with reference to the accompanying drawings in which like characters represent like parts throughout the drawings, wherein:

FIG. 1 illustrates a block diagram of an embodiment of an ultrasound imaging system having a haptic guidance system, in accordance with aspects of the present disclosure;

FIG. 2 illustrates a perspective view of an embodiment of an ultrasound probe of the ultrasound imaging system having the haptic guidance system of FIG. 1, in accordance with aspects of the present disclosure;

FIG. 3 illustrates a schematic of the ultrasound probe of FIG. 2 showing an embodiment of a vibration sequence of vibration actuators of the haptic guidance system of FIG. 1 to guide movement of the ultrasound probe along an X-axis, in accordance with aspects of the present disclosure;

FIG. 4 illustrates a schematic of the ultrasound probe of FIG. 2 showing another embodiment of a vibration sequence of the vibration actuators of the haptic guidance system of FIG. 1 to guide movement of the ultrasound probe along the X-axis, in accordance with aspects of the present disclosure;

FIG. 5 illustrates a schematic of the ultrasound probe of FIG. 2 showing an embodiment of a vibration sequence of the vibration actuators of the haptic guidance system of FIG. 1 to guide movement of the ultrasound probe along a Y-axis, in accordance with aspects of the present disclosure;

FIG. 6 illustrates a schematic of the ultrasound probe of FIG. 2 showing another embodiment of a vibration sequence of the vibration actuators of the haptic guidance system of FIG. 1 to guide movement of the ultrasound probe along the Y-axis, in accordance with aspects of the present disclosure;

FIG. 7 illustrates a schematic of the ultrasound probe of FIG. 2 showing an embodiment of a vibration sequence of the vibration actuators of the haptic guidance system of FIG. 1 to guide compression movement the ultrasound probe along a Z-axis, in accordance with aspects of the present disclosure;

FIG. 8 illustrates a schematic of the ultrasound probe of FIG. 2 showing another embodiment of a vibration sequence of the vibration actuators of the haptic guidance system of FIG. 1 to guide compression movement the ultrasound probe along the Z-axis, in accordance with aspects of the present disclosure;

FIG. 9 illustrates a schematic of the ultrasound probe of FIG. 2 showing an embodiment of a vibration sequence of the vibration actuators of the haptic guidance system of FIG. 1 to guide tilting movement of the ultrasound probe along an elevation direction, in accordance with aspects of the present disclosure;

FIG. 10 illustrates a schematic of the ultrasound probe of FIG. 2 showing an embodiment of a vibration sequence of the vibration actuators of the haptic guidance system of FIG. 1 to guide rocking movement of the ultrasound probe along an azimuth direction, in accordance with aspects of the present disclosure;

FIG. 11 illustrates a schematic of the ultrasound probe of FIG. 2 showing an embodiment of a vibration sequence of vibration actuators of the haptic guidance system of FIG. 1 to guide rotation movement of the ultrasound probe, in accordance with aspects of the present disclosure; and

FIG. 12 illustrates a flow diagram of an embodiment of a method for guiding movement of the ultrasound probe using the haptic guidance system of FIG. 1, in accordance with aspects of the present disclosure.

### DETAILED DESCRIPTION

One or more specific embodiments will be described below. In an effort to provide a concise description of these embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

When introducing elements of various embodiments of the present disclosure, the articles “a,” “an,” “the,” and “said” are intended to mean that there are one or more of the elements. The terms “comprising,” “including,” and “having” are intended to be inclusive and mean that there may be additional elements other than the listed elements. Furthermore, any numerical examples in the following discussion are intended to be non-limiting, and thus additional numerical values, ranges, and percentages are within the scope of the disclosed embodiments.

As mentioned above, ultrasound imaging systems typically include an ultrasound probe that is applied to a patient's body. Ultrasound imaging systems may be operated by skilled technicians trained in locating the target region of interest on the patient to be imaged and the target scan plan, generally by using the ultrasound image data as guidance. A challenge in the field of ultrasound to widespread growth and adoption of ultrasound imaging systems in every physician's office and throughout the developing world is the lack of skilled sonographers (e.g., technicians) or operators capable of performing such interpretation of the data and manipulation of the probe. As such, it is recognized that it may be desirable for the ultrasound imaging system to provide guidance to less skilled users so that they may obtain proper ultrasound images for diagnosis. Therefore, an operator-independent ultrasound imaging system may consist of a mix of automation of some operational steps that are conventionally performed by an operator and machine guidance of other operational steps. For example, in the operator-independent ultrasound imaging system, many of the buttons or selections that an operator would conventionally adjust may be automated. Additionally, to assist the operator with orienting the ultrasound probe physically into a position to acquire the target scan plane of the target anatomy, described herein are ultrasound imaging systems and ultrasound probes having an integrated haptic feedback system for guided ultrasound probe placement. Such automation and guidance may allow novice, or less skilled, users to obtain successful ultrasound scans.

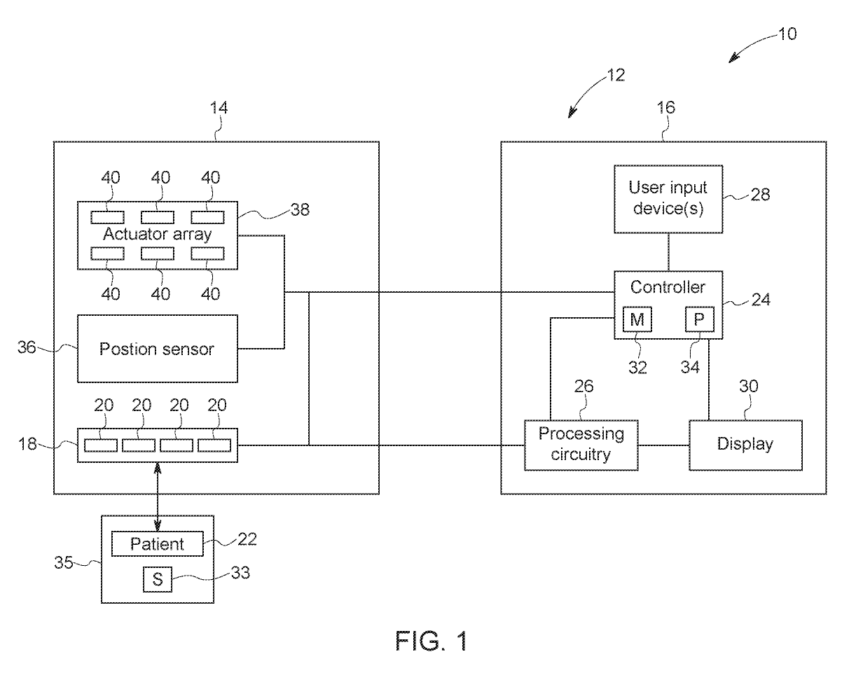

With the foregoing in mind, FIG. 1 illustrates a block diagram of an embodiment of an ultrasound imaging system 10 having a haptic guidance system 12 (e.g., haptic feedback system) that may be used to provide haptic feedback to an operator of ultrasound imaging system 10 to guide placement of an ultrasound probe 14 of the ultrasound imaging system 10. In the illustrated embodiment, the ultrasound system 10 is a digital acquisition and beam former system, but in other embodiments, the ultrasound system 10 may be any suitable type of ultrasound system, not limited to the illustrated type. The ultrasound system 10 may include the ultrasound probe 14 and a workstation 16 (e.g., monitor, console, user interface) which may control operation of the ultrasound probe 14 and may process image data acquired by the ultrasound probe 14. The ultrasound probe 14 may be coupled to the workstation 16 by any suitable technique for communicating image data and control signals between the ultrasound probe 14 and the workstation 16 such as a wireless, optical, coaxial, or other suitable connection.

The ultrasound probe 14 contacts the patient during an ultrasound examination. The ultrasound probe 14 may include a patient facing or contacting surface that includes a transducer array 18 having a plurality of transducer elements 20. Each individual transducer element 20 may be capable of converting electrical energy into mechanical energy for transmission and mechanical energy into electrical energy for receiving. It should be noted that the transducer array 18 may be configured as a two-way transducer capable of transmitting ultrasound waves into and receiving such energy from a subject or patient 22 during operation when the ultrasound probe 14 is placed in contact with the patient 22. More specifically, the transducer elements 20 may convert electrical energy from the ultrasound probe 14 into ultrasound waves (e.g., ultrasound energy, acoustic waves) and transmit the ultrasound waves into the patient 22. The ultrasound waves may be reflected back toward the transducer array 18, such as from tissue of the patient 22, and the transducer elements 20 may convert the ultrasound energy received from the patient 22 (reflected signals or echoes) into electrical signals for transmission and processing by the ultrasound probe 14 and the workstation 16 to provide an ultrasound image that may be analyzed. The number of transducer elements 20 in the transducer array 18 and the frequencies at which the transducer elements 20 operate may vary depending on the application.

As previously discussed, the ultrasound probe 14 is communicatively coupled to the workstation 16 of the ultrasound imaging system 10 to facilitate image collection and processing. As will be appreciated, the workstation 16 may include a number of elements to control operation of the ultrasound probe 14, facilitate placement guidance of the ultrasound probe 14, and facilitate production of ultrasound images. For instance, as illustrated, the workstation 16 may include a controller 24, processing circuitry 26, one or more user input devices 28, and a display 20. In certain embodiments, the workstation 16 may include additional elements not shown in FIG. 1, such as additional data acquisition and processing controls, additional image display panels, multiple user interfaces, and so forth.

The controller 24 may include a memory 32 and a processor 34. In some embodiments, the memory 32 may include one or more tangible, non-transitory, computer-readable media that store instructions executable by the processor 34 and/or data to be processed by the processor 34. For example, the memory 32 may include random access memory (RAM), read only memory (ROM), rewritable non-volatile memory such as flash memory, hard drives, optical discs, and/or the like. Additionally, the processor 34 may include one or more general purpose microprocessors, one or more application specific processors (ASICs), one or more field programmable logic arrays (FPGAs), or any combination thereof. The controller 24 may control transmission of the ultrasound waves into the patient 22 via the transducer array 18. Additionally, the controller 24 may be part of the haptic guidance system 12 of the ultrasound system 10 and may control other elements of the haptic guidance system 12 to guide placement of the ultrasound probe 14 for acquiring ultrasound images of the target scan plane of the target anatomy of the patient 22, as discussed in greater detail below.

The processing circuitry 26 may include receiving and conversion circuitry. The processing circuitry 26 may receive the electrical signal data from the transducer array 18 of the ultrasound probe 14 representing reflected ultrasound energy returned from tissue interfaces within the patient 22. The processing circuitry 26 may process the data from the transducer array 18, such as correcting for noise artifacts, or the like. The processing circuitry 26 may then convert the signal data into an ultrasound image for presentation via the display 30. The controller 24 may cause display of the ultrasound image or images produced by the processing circuitry 26 from the signal data received from the transducer array 18 of the ultrasound probe 14.

In operation, the controller 24 may receive a signal indicative of a target anatomy of the patient 22 and/or a target scan plane of the target anatomy via the one or more user input devices 28 of the workstation 16. The one or more user input devices 28 may include a keyboard, a touchscreen, a mouse, buttons, switches, or other devices suitable to allow the operator to input a the target anatomy and/or the desired scan plane of the target anatomy. Based on the target anatomy and/or the target scan plane of the target anatomy, the controller 24 may output a signal to the transducer array 18 of the ultrasound probe 14 indicative of an instruction to convert the electrical energy from the ultrasound probe 14 into ultrasound waves and transmit the ultrasound waves into the patient 22 and to detect the ultrasound energy that is reflected back from the tissue interfaces within the patient 22.

The controller 24 may determine that the ultrasound probe 14 is in an appropriate position and/or orientation (e.g., target position and/or orientation) to sufficiently image the target anatomy in the target scan plane. In some embodiments, the haptic guidance system 12 of the ultrasound imaging system 10 may include sensing mechanisms to detect where the patient is and to determine the position and/or orientation of the ultrasound probe 14 with respect to the patient 22. That is, the haptic guidance system 12 may include one or more patient position sensors 33, such as weight sensors, contact sensors, cameras, or other suitable sensing mechanisms, that may be disposed about the imaging space 35 of the ultrasound imaging system 10 on or in which the patient 22 is positioned during ultrasound imaging, or at any other suitable position about the ultrasound imaging system 10 suitable for detecting the position of the patient 22. The patient position sensor 33 may be communicatively coupled to the controller 24 via a wired or wireless connection and may send one or more signals to the controller 24 indicative of the position of the patient about the imaging space 35. Additionally or alternatively, in some embodiments, a position of the patient 22 relative to the imaging space 35 or the ultrasound imaging system 10 may be determined based at least in part on received demographic information, such as height and weight, of the patient 22. For example, such demographic information of the patient 22 may be compared to lookup tables stored in the memory 32 to determine a relative position of the patient 22.

Based at least in part on the one or more signals received from the patient position sensor 33 and/or the received demographic information of the patient 22, the controller may determine, develop, or update a patient-centric coordinate system or map that may be used to track position of the ultrasound probe 14 relative to the patient 22. For example, a general patient coordinate system based on the imaging space 35 may be stored in the memory 32 and the controller 24 may update the general patient coordinate system based at least in part on the signals received from the patient position sensor 33 and/or the received demographic information of the patient 22 to generate the patient-centric coordinate system. As another example, the controller 24 may develop a new patient-centric coordinate system for each patient based at least in part on the signals received from the patient position sensor 33 and/or the received demographic information of the patient 22. Additionally or alternatively, in some embodiments, the patient position sensor 33 may include an ultrasound probe with integrated position tracking. In such embodiments, an operator could follow a procedure while using the ultrasound probe with integrated position tracking to place the probe on key landmarks or drag the probe across the surface of the patient until the coordinate system is developed. Additionally or alternatively, in some embodiments, the patient-centric coordinate system may be derived based at least in part on image data received from one or more cameras of the ultrasound imaging system 10 that may be used to measure or determine patient position and/or the live ultrasound image data from the ultrasound probe 14. The patient-centric coordinate system may allow the controller 24 to determine a relative position of the target anatomy within the patient 22 and to determine when the ultrasound probe 14 is positioned appropriately to sufficiently image the target anatomy in the target scan plane. Additionally, the patient-centric coordinate system may be compared or fused with a model of general body anatomy, which may be stored in the memory 32.

The haptic guidance system 12 of the ultrasound imaging system 10 may additionally include a probe position sensor 36 for detecting the position and/or orientation of the ultrasound probe 14. The probe position sensor 36 may be disposed about the ultrasound probe 14 and may be a position sensor, an orientation sensor, such as a gyroscope, inertial tracking mechanism, electromagnetic tracking, optical tracking, or any other suitable sensor that may allow for detection of a current position and/or orientation of the ultrasound probe 14. The probe position sensor 36 may be communicatively coupled to the controller 24 via a wired or wireless connection and may send one or more signals to the controller 24 indicative of a current position and/or orientation of the ultrasound probe 14. The controller 24 may compare the current position of the ultrasound probe 14, based at least in part on the signals received from the probe position sensor 36, to the patient-centric coordinate system developed based at least in part on the signals received from the patient position sensor 33 and/or the live ultrasound image data. Based on the comparison, the controller 24 may determine whether the ultrasound probe 14 is in the appropriate position and/or orientation to sufficiently image the target anatomy in the target scan plane. As such, the live ultrasound image data may be used by the controller 24 as feedback to determine if the ultrasound probe 14 is in the appropriate position and orientation. The patient-centric coordinate system and the ultrasound image data may be stored in the memory 32.

In some embodiments, the controller 24 may control the transducer array 18 and the processing circuitry 26 to obtain and generate ultrasound images while the controller 24 is determining whether the ultrasound probe 14 is in the appropriate position and/or orientation to sufficiently image the target anatomy in the target scan plane. As such, the live ultrasound image data may be used by the controller 24 as feedback in determining if the ultrasound probe is in the appropriate position and/or orientation to sufficiently image the target anatomy in the target scan plane. Once the controller 24 determines that ultrasound probe 14 is in the appropriate position and/or orientation to sufficiently image the target anatomy in the target scan plane, the controller 24 may cause display of a notification, such as a visual or audible notification, and/or the controller 24 may send a signal to the vibration actuators 40 of the ultrasound probe 14 indicative of a particular pulse pattern indicating to the operator of the ultrasound probe 14 that the ultrasound probe 14 is in the appropriate position and/or orientation. Additionally or alternatively, in some embodiments, the controller 24 may cause automatic storage in the memory 32 of the live ultrasound image data once it is determined that the ultrasound probe is in the appropriate position and/or orientation. Additionally or alternatively, in some embodiments, if the controller 24 determines that the ultrasound probe 14 is in the appropriate position and/or orientation, the controller 24 may send a signal to the processing circuitry 26 indicative of an instruction to convert the data received from the transducer array 18 of the ultrasound probe 14 into an ultrasound image and a signal to the display 30 indicative of an instruction to display the ultrasound image. Additionally or alternatively, in some embodiments, the controller 24 may not cause the transducer array 18 to produce and transmit the ultrasound waves into the patient 22 and detect the reflected ultrasound energy until it is determined that the ultrasound probe 14 is in the appropriate position and/or orientation to sufficiently image the target anatomy in the target scan plane.

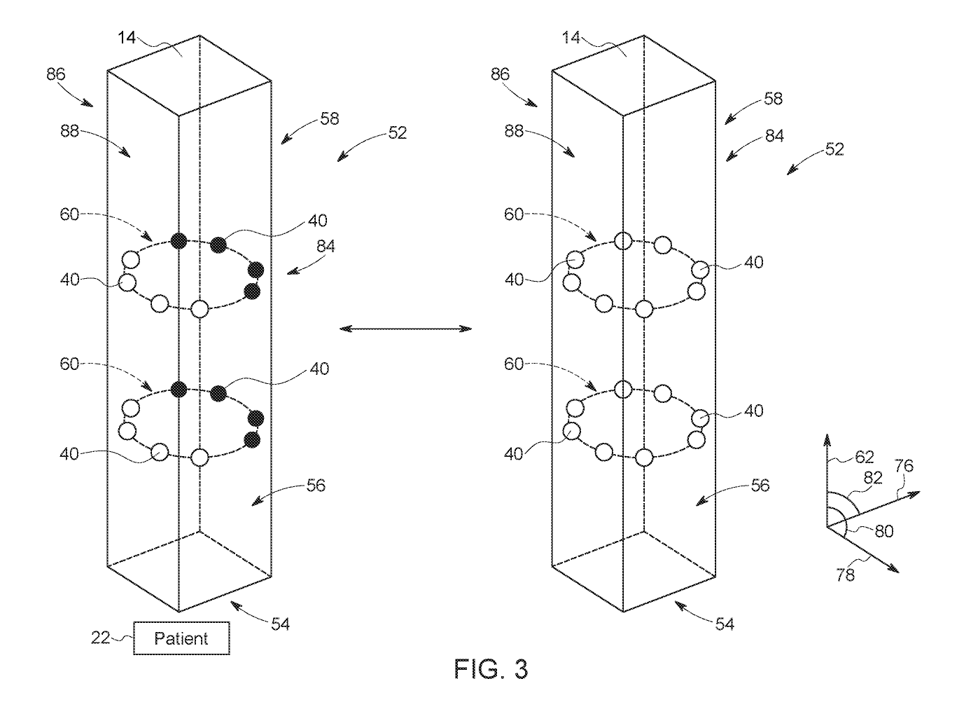

If the controller 24 determines that the ultrasound probe 14 is not in the appropriate position and/or orientation to sufficiently image the target anatomy in the target scan plane, the controller 24 may guide the operator of the ultrasound probe 14 to move the ultrasound probe 14 into the appropriate position. As such, the haptic guidance system 12 may include an actuator array 38 including multiple vibration actuators 40 (e.g., haptic actuators) distributed among a grip area on some or all sides of a handle of the ultrasound probe 14. The controller 24 may send one or more signals to one or more of the vibration actuators 40 to cause the vibration actuators 40 to vibrate in a particular pattern indicative of a particular target movement of the ultrasound probe 14 to guide an operator to move the ultrasound probe 14 into the appropriate position. The vibration actuators 40 may be driven by the controller 24 to guide positioning of the ultrasound probe 14 in six degrees-of-freedom (DOF) (i.e., spatial position and angular orientation) by using various vibration patterns, discussed in greater detail with reference to FIGS. 3-11. By placing the vibration actuators 40 on some or all sides of the ultrasound probe 14, multiple grip styles may be supported. Once the operator has located the ultrasound probe 14 in the appropriate position over the target anatomy, the operator may be further guided by the controller 24 to make fine adjustments to the ultrasound probe 14 position and/or orientation to acquire the target scan plane. As discussed above, in some embodiments, the controller 24 of the ultrasound imaging system 10 may use the live (e.g., real-time) ultrasound image data and image processing technology to detect the anatomy and guide this fine positioning while comparing the live ultrasound image data to the target scan plane.

As such, the haptic guidance system 12 of the ultrasound imaging system 10 may provide guidance for the placement of the ultrasound probe 14 relative to the patient 22 through haptic feedback incorporated directly into the handle of the ultrasound probe 14. Such haptic feedback and guidance may appeal to an alternate sense perception than sight, which may provide more intuitive guidance to an operator instead of visual cues as the operator is already focused on viewing the ultrasound image, or voice guidance as the operator may be trying to communicate with the patient. For example, visual sense perception of the operator is already used to comprehend the live ultrasound image, to operate the workstation 16, to communicate with the patient 22, and in other events taking place near the ultrasound imaging system 10. Additionally, auditory sense perception is used for communicating with the patient 22. As tactile sense perception of the operator is already used for moving the ultrasound probe 13, such that the operator is not looking at the ultrasound probe 14, but rather is visually focused on the workstation 16 and the live ultrasound image, use of the vibration actuators 40 in the ultrasound probe 14 for probe positioning may take advantage of an underutilized sense perception. By using the actuator array 38 of the multiple vibration actuators 40, the position and orientation of the ultrasound probe 14 may be guided in all six degrees-of-freedom. As such, the haptic guidance system 12 may allow for more efficient and effective use of the ultrasound imaging system 10 by novice or less-skilled operators and thus, may increase operator independence of the ultrasound imaging system 10.

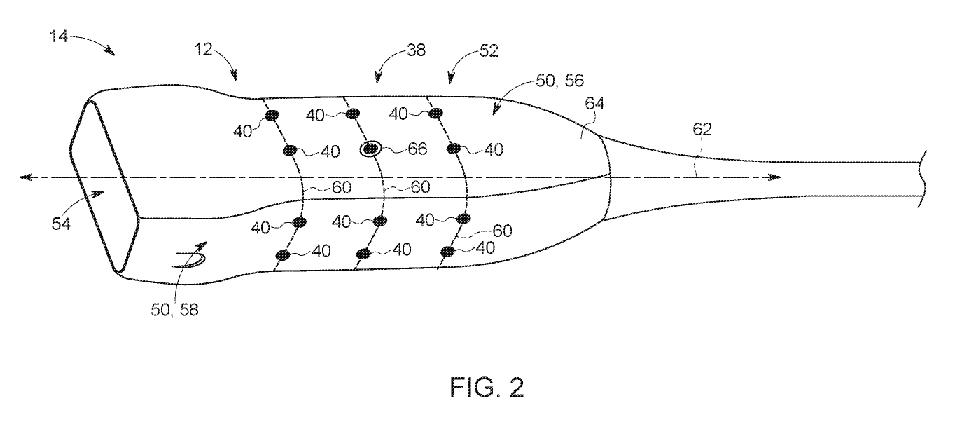

FIG. 2 illustrates a perspective view of the ultrasound probe 14 showing an embodiment of the placement of the vibration actuators 40 of the actuator array 38 of the haptic guidance system 12. As previously discussed, the vibration actuators 40 may be directed to vibrate in particular vibration patterns (e.g., actuation patterns, excitation patterns) representing particular movements of ultrasound probe 14 relative to six degrees-of-freedom in which the ultrasound probe 14 can be moved. The vibration patterns of the vibration actuators 40 may guide the operator to position the ultrasound probe 14 in the appropriate position and/or orientation to sufficiently image the target anatomy in the target scan plane. The multiple vibration actuators 40 of the actuator array 38 may be distributed on some or all sides 50 of a handle 52 of the ultrasound probe 14. That is, the handle 52 of the ultrasound probe 14 may include the vibration actuators 40 on each side 52 or on only a subset of the available sides 50 of the handle 52. The sides 50 may form the handle 52 and may be oriented perpendicular to a patient-facing surface 54 (e.g., face) of the ultrasound probe 14. The illustrated embodiment shows six vibration actuators 40 on a front side 56 (e.g., upper surface) and four vibration actuators 40 on a first side 58 (e.g., left side) of the handle 52 of the ultrasound probe 14. Additionally, though not shown in the illustrated embodiment, the ultrasound probe 14 may include corresponding vibration actuators 40 on the opposing surfaces, such as a back surface and an opposite side (e.g., right side), of the handle 52. As such, the actuator array 28 may support and allow for positioning guidance for multiple grip styles.

Each side 50 of the handle 52 of the ultrasound probe 14 may include two or more vibration actuators 40. In some embodiments, the locations of the vibration actuators 40 about the handle 52 may create two or more parallel rings 60 of vibration actuators 40 axially offset from each other about an axial axis 62 of the ultrasound probe 14, with each ring 60 of vibration actuators 40 having vibration actuators 40 disposed on each side 50 of the handle 52. As will be appreciated, though the term “side” is used herein, this term should be understood broadly to encompass any facings or surfaces of the handle 52 of the ultrasound probe 14. For example, in the context of a cylindrical or ovoid handle, vibration actuators may be provided around the handle such that different vibration actuators face different directions adjacent or opposite one another (i.e., face different radial directions with respect to the handle), though no structurally distinct or discernible side may be geometrically identifiable.

The vibration actuators 40 may be integrated into the handle 52. That is, the vibration actuators 40 may each be disposed below a housing 64 of the ultrasound probe 14. That is, although the vibration actuators 40 are illustrated as disposed outside of or on an outer surface of the housing 64 for visual clarity, the vibration actuators 40 may be disposed within the handle 52 of the ultrasound probe 14 adjacent to the housing 64. In some embodiments, each vibration actuator 40 may be housed in a shell 66 such that each vibration actuator 40 is at least partially decoupled from the housing 64. For example, each vibration actuator 40 may be surrounded by a rubber ring, covering, or other suitable structure, such that the vibrations are spatially contained to the location of the individual vibration actuators 40 and minimal vibration is passed through the housing 64 and thus, providing location-specific vibrations. Location-specific vibration of each vibration actuator 40 may allow the operator to better sense the vibration patterns that may be used to guide the operator to position the ultrasound probe 14 into the appropriate position for capturing the target scan plane of the target anatomy (e.g., anatomy of interest).

In some embodiments, the guidance method may be varied, as may the actuation mechanisms. The embodiments described herein may facilitate positioning of the ultrasound probe 14 based on haptic feedback in all six degrees-of-freedom (i.e., three denoting spatial position and three denoting orientation, such as angular orientation). In some embodiments, the concepts described herein with respect to the actuator array 38 of the vibration actuators 40 and various vibration patterns may be extended to other actuation mechanisms, such as small motion actuators oriented perpendicular to the probe housing that protrude in and out at each actuation location, small motion actuators mounted in a shear orientation about the probe housing (e.g., parallel with the housing) that drag across the hand of the operator, small electrical currents coupled into the hand through electrodes at each of these locations, small puffs of air at each of these locations, small protrusions in and out of a deformable membrane due to pressurization and depressurization of fluid filled chambers, or the like. As an example of the various possible guidance methods, a simpler haptic feedback approach may fuse visual feedback or voice commands with a single vibration that is provided as the ultrasound probe 14 is moved into the appropriate position, for example a pulse that becomes faster or slower as the ultrasound probe 14 is moved into the appropriate position. In some embodiments, the guidance method may include a fusion of visual feedback, auditory commands, and/or haptic feedback using the vibration actuators, where motion in some degrees-of-freedom may be indicated through haptic feedback and motion in other degrees-of-freedom may be indicated through visual and/or auditory mechanisms.

The controller 24 of the ultrasound imaging system 10 may cause vibration of the vibration actuators 40 integrated into the handle 52 of the ultrasound probe 14 to guide the operator into the appropriate position and orientation to image the target anatomy in the target scan plane. The particular vibration patterns (e.g., excitation patterns) may indicate to the operator how to move the ultrasound probe 14 to acquire an image of the target anatomy in the target scan plane. Particular vibration patterns may indicate to the operator to move the ultrasound probe 14 in a particular direction, to tilt or rock the ultrasound probe 14, to rotate the ultrasound probe 14, and/or to compress or decompress the ultrasound probe 14 toward or away from the patient 22. In some embodiments, the operator may be guided to move the ultrasound probe 14 in more than one degree-of-freedom simultaneously by applying more complex vibration patterns.

For each vibration pattern discussed below, the vibrations of the vibration actuators 40 may continue until the operator has moved the ultrasound probe 14 to the appropriate position. In some embodiments, to guide a speed of movement of the ultrasound probe 14 by the operator, a repetition rate of the vibration pattern may vary or modulate. For example, in some embodiments, the repetition rate may be faster when the ultrasound probe 14 is farther away from the appropriate position, and then may slow down as the ultrasound probe 14 is moved closer to appropriate position to image the target anatomy and/or the target scan plane. In some embodiments, the repetition rate may be slower when the ultrasound probe 14 is farther away from the appropriate position, and then may increase in speed as the ultrasound probe 14 is moved closer to appropriate position to image the target anatomy and/or the target scan plane. Additionally or alternatively, an intensity of the vibrations may vary. For example, in some embodiments, the vibrations may be stronger when the ultrasound probe 14 is farther away from the appropriate position, and then may decrease in intensity when the ultrasound probe 14 is moved closer to the appropriate position to image the target anatomy and/or the target scan plane. In some embodiments, the vibrations may be lower intensity when the ultrasound probe 14 is farther away from the appropriate position, and then may increase in intensity when the ultrasound probe 14 is moved closer to the appropriate position to image the target anatomy and/or the target scan plane. As such, the speed and/or the intensity of the vibrations of the vibration actuators 40 may operate as a closed feedback loop to more accurately and efficiently guide the operator to adjust the ultrasound probe 14 into the appropriate position.

With the foregoing in mind, examples of the vibration patterns that may be used to guide particular movement of the ultrasound probe 14 are discussed below with reference to FIGS. 3-11. The particular vibration patterns may guide the operator to move the ultrasound probe 14 in six degrees-of-freedom, including movement right and left, forward and backward, compression and decompression, tilt along an elevation direction, rock along an azimuth direction, and rotation. In FIGS. 3-11, the vibration patterns are shown as a series of vibration pulses, where open vibration actuators 40 indicate still or currently non-vibrating actuators and solid or filled vibration actuators 40 indicate currently vibrating actuators. Additionally, movement of the ultrasound probe 14 in six degrees-of-freedom is discussed with reference to the axial axis or direction 62 (e.g., Z-axis), an X-axis 76, a Y-axis 78, an elevation angle 80, and an azimuth angle 82. For reference, it should be noted that with regard to FIGS. 3-11, directional motion is discussed in terms of the operator being positioned within the page such that forward movement describes movement out of the page and backward movement describes movement into the page. Additionally, with regard to FIGS. 3-11, while the vibration patterns are described in terms of indicating movement toward the pulsing or longer vibrations or to follow the vibration pattern, in some embodiments, the vibration patterns may indicate movement away from the pulsing or longer vibration or to move opposite the vibration pattern based on operator preferences.

FIG. 3 illustrates an embodiment of a simple vibration pattern (denoted by iteration between the left-hand and right-hand depictions, as denoted by the two-way arrow) of the vibration actuators 40 that may be used to guide movement of the ultrasound probe along the X-axis 76. For example, the illustrated vibration pattern may be used to guide movement of the ultrasound probe 14 in a linear direction e.g., toward a first side 58 (e.g., left side). That is, the haptic guidance system 12 may cause the vibration actuators 40 on a first half 84 (e.g., a first half about the axial axis 62 extending from the patient-facing surface 54 to an opposite non-patient-facing surface, left half) of the handle 52 of the ultrasound probe 14 to pulse, as shown between the left-hand and right-hand depictions of FIG. 3. The first half 84 of the ultrasound probe 14 may include half of some or all of the rings 60 of vibration actuators 40 disposed in the first half 84. Such pulsing on and off of the vibration actuators 40 may indicate to the operator of the ultrasound probe 14 to move the ultrasound probe 14 in a first direction, the direction of the pulsing. In some embodiments, the pulses of the vibration actuators 40 on the first half 84 of the ultrasound probe 14 may be short pulses, long pulses, or a combination thereof.

In some embodiments, the pulse length, pulse repetition rate, and/or pulse intensity may vary to indicate a distance from and/or to guide a speed of the movement of the ultrasound probe 14 to the appropriate position and/or orientation to sufficiently image the target anatomy in the target scan plane. For example, the pulse repetition rate of the vibration actuators 40 on the first half 84 of the ultrasound probe 14 may be faster when the ultrasound probe 14 is farther away from the appropriate position, and then may slow down as the ultrasound probe 14 is moved closer to the appropriate position to image the target anatomy and/or the target scan plane, or vice versa. Additionally or alternatively, an intensity of the vibrations may be stronger when the ultrasound probe 14 is farther away from the appropriate position, and then may decrease in intensity when the ultrasound probe 14 is moved closer to the appropriate position to image the target anatomy and/or the target scan plane, or vice versa. Further, additionally or alternatively, the pulse length of the vibrations of the vibration actuators 40 of the first half 84 of the ultrasound probe 14 may be longer when the ultrasound probe 14 is farther away from the appropriate position, and then may decrease in length when the ultrasound probe 14 is moved closer to the appropriate position to image the target anatomy and/or the target scan plane, or vice versa. Thus, the simple pulsing of the vibration actuators 40 of the first half 84 of the ultrasound probe 14 caused by the controller 24 of the haptic guidance system 12 may guide the operator of the ultrasound probe 14 to move the ultrasound probe 14 in the first direction along the X-axis 76.

The opposite vibration pattern may be used to guide movement of the ultrasound probe 14 in the opposite direction along the X-axis 76. That is, the haptic guidance system 12 may cause the vibration actuators 40 on a second half 86 (e.g., a half opposite the first half about the axial axis 62 extending from the patient-facing surface 54 to an opposite non-patient-facing surface, right half) of the handle 52 of the ultrasound probe 14 to pulse, indicating to the operator of the ultrasound probe 14 to move the ultrasound probe 14 toward a second side 88 (e.g., right side) of the ultrasound probe 14 in a second direction, the direction of the pulses. The second half 86 of the ultrasound probe 14 may include the half of some or all of the rings 60 of vibration actuators 40 in the second half 86. In some embodiments, the pulse length, pulse repetition rate, and/or pulse intensity may vary as discussed above to indicate a distance from and/or to guide a speed of the movement of the ultrasound probe 14 to the appropriate position to sufficiently image the target anatomy in the target scan plane.

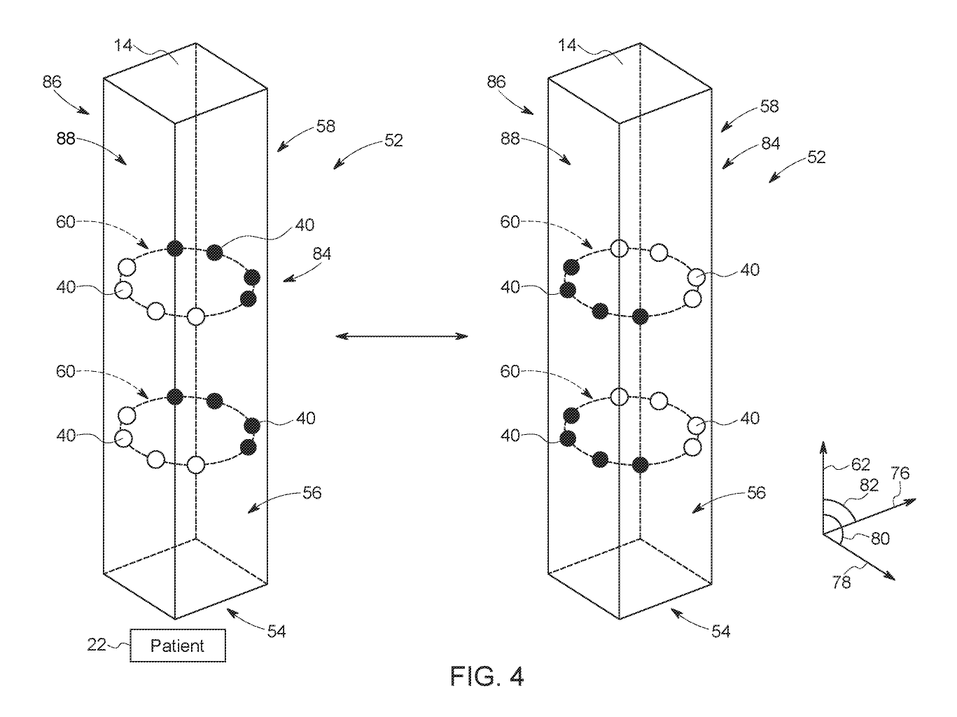

FIG. 4 illustrates an alternate vibration pattern that may be used to guide movement of the ultrasound probe 14 along the X-axis 76. For example, the illustrated vibration pattern may be used to guide movement of the ultrasound probe 14 to the left toward the first side 58. The haptic guidance system 22, in some embodiments, may cause the vibration actuators 40 on the second half 86 of the handle 52 of the of the ultrasound probe 14 to pulse in one or more short pulses, and then cause the vibration actuators 40 on the first half 84 of the ultrasound probe 14 to pulse in one or more long pulses indicating to the operator to move the ultrasound probe 14 in the first direction, the direction of the long pulses. As such, the vibration pattern may include alternating short pulses on the second half 86 of the ultrasound probe 14 and long pulses on the first half 84 of the ultrasound probe 14. In some embodiments, as discussed above with reference to FIG. 3, the pulse speed and/or pulse intensity of the vibrations of the vibration actuators 40 may be varied as the ultrasound probe 14 is moved closer to appropriate position to image the target anatomy and/or the target scan plane to indicate proximity and/or speed of movement to the operator of the ultrasound probe 14.

The opposite vibration pattern may be used to guide movement of the ultrasound probe 14 in the opposite direction along the X-axis 76. That is, the haptic guidance system 12 may cause the vibration actuators 40 on the first half 84 of the handle 52 of the ultrasound probe 14 to pulse in one or more short pulses, and then cause the vibration actuators 40 on the second half 86 of the handle 52 of the ultrasound probe 14 to pulse in one or more long pulses indicating to the operator to move the ultrasound in the second direction, the direction of the long pulses. As such, the vibration pattern may include alternating short pulses on the first half 84 of the ultrasound probe 14 and long pulses on the second half 86 of the ultrasound probe 14. In some embodiments, as discussed above, the pulse speed and/or pulse intensity of the vibrations of the vibration actuators 40 may be varied as the ultrasound probe 14 is moved closer to appropriate position to image the target anatomy and/or the target scan plane to indicate proximity and/or speed of movement to the operator of the ultrasound probe 14.

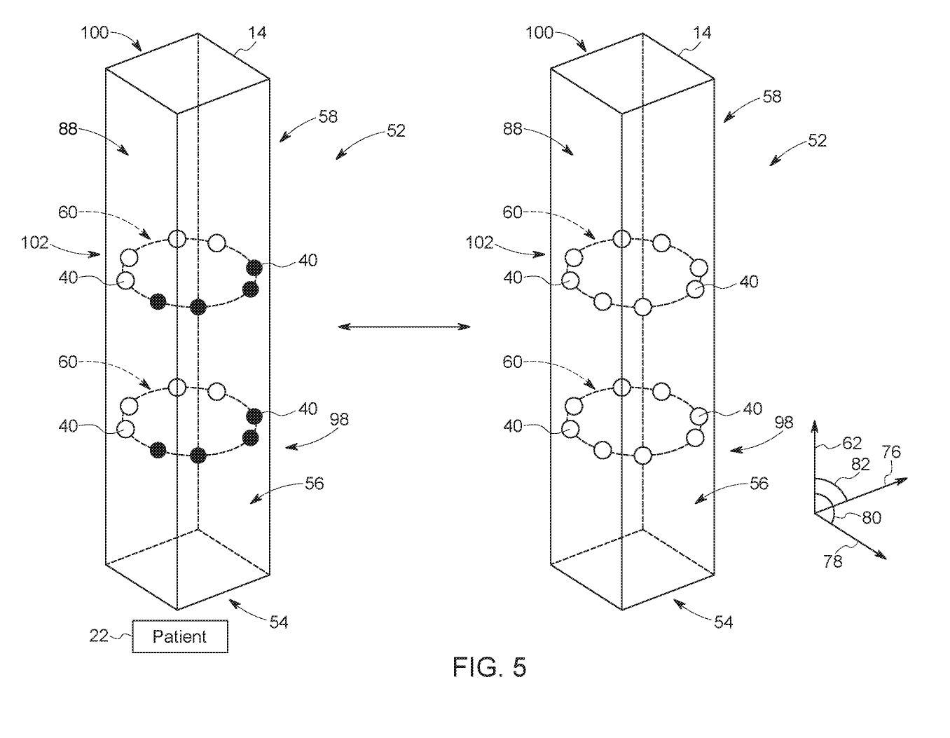

Similar vibration patterns may be used to guide movement of the ultrasound probe 14 forward (e.g., away from the operator) and backward (e.g., toward the operator) along the Y-axis 78, as illustrated in FIG. 5. For example, the illustrated vibration pattern may be used to guide movement of the ultrasound probe 14 forward away from the operator and toward the front side 56. The haptic guidance system 12, in some embodiments, may cause the vibration actuators 40 on a front half 98, of the handle 52 of the ultrasound probe 14 to pulse, indicating to the operator of the ultrasound probe 14 to move the ultrasound probe 14 toward the front side 56 of the ultrasound probe 14 in the direction of the pulses. The front half 98 of the ultrasound probe 14 may include the front half of some or all of the rings 60 of vibration actuators 40. In some embodiments, the pulse length, pulse repetition rate, and/or pulse intensity may vary, as discussed above with reference to FIG. 3, to indicate a distance from and/or to guide a speed of the movement of the ultrasound probe 14 to the appropriate position to sufficiently image the target anatomy in the target scan plane.

The opposite vibration pattern may be used to guide movement of the ultrasound probe 14 in the opposite direction (e.g., backward) toward the operator and a back side 100 (e.g., side opposite the front side) of the ultrasound probe 14 along the Y-axis 78. That is, the haptic guidance system 12 may cause the vibration actuators 40 on a back half 102 of the handle 52 of the ultrasound probe 14 to pulse, indicating to the operator of the ultrasound probe 14 to move the ultrasound probe 14 backward toward the back side 100 of the ultrasound probe 14 in the direction of the pulses. The back half 102 of the ultrasound probe 14 may include the back half of some or all of the rings 60 of vibration actuators 40. In some embodiments, the pulse length, pulse repetition rate, and/or pulse intensity may vary as discussed above to indicate a distance from and/or to guide a speed of the movement of the ultrasound probe 14 to the appropriate position to sufficiently image the target anatomy in the target scan plane.

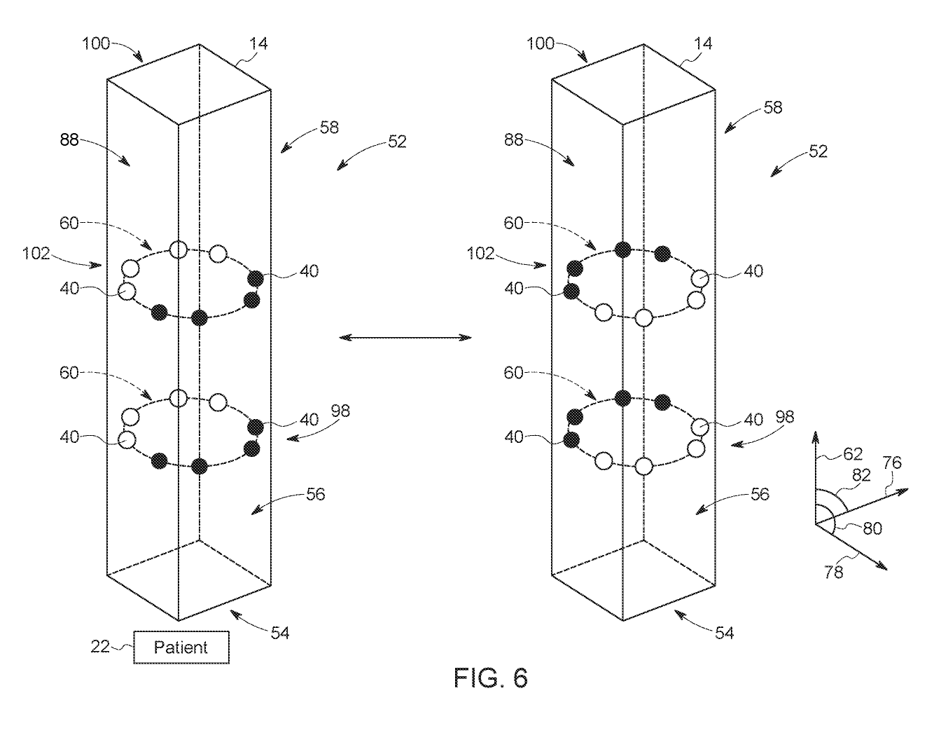

FIG. 6 illustrates an alternate vibration pattern that may be used to guide movement of the ultrasound probe 10 forward (e.g., away from the operator) and backward (e.g., toward the operator) along the Y-axis 78. For example, the illustrated vibration pattern may be used to guide movement of the ultrasound probe 14 forward away from the operator and toward the front side 56. The haptic guidance system 12, in some embodiments, may cause the vibration actuators 40 on the back half 102 of the handle 52 of the ultrasound probe 14 to pulse in one or more short pulses, and then cause the vibration actuators 40 on the front half 98 of the handle 52 of the ultrasound probe 14 to pulse in one or more long pulses indicating to the operator to move the ultrasound probe 14 in the direction of the long pulses. As such, the vibration pattern may include alternating short pulses on the back half 102 of the ultrasound probe 14 and long pulses on the front half 98 of the ultrasound probe 14. In some embodiments, as discussed above with reference to FIG. 3, the pulse speed and/or pulse intensity of the vibrations of the vibration actuators 40 may be varied as the ultrasound probe 14 is moved closer to appropriate position to image the target anatomy and/or the target scan plane to indicate proximity and/or speed of movement to the operator of the ultrasound probe 14.

The opposite vibration pattern may be used to guide movement of the ultrasound probe 14 in the opposite direction (e.g., backward) toward the operator and the back side 100 along the Y-axis 78. That is, the haptic guidance system 12 may cause the vibration actuators 40 on the front half 98 of the handle 52 of the ultrasound probe 14 to pulse in one or more short pulses, and then cause the vibration actuators 40 on the back half 102 of the handle 52 of the ultrasound probe 14 to pulse in one or more long pulses indicating to the operator to move the ultrasound in the direction of the long pulses. As such, the vibration pattern may include alternating short pulses on the front half 98 of the ultrasound probe 14 and long pulses on the back half 102 of the ultrasound probe 14. In some embodiments, as discussed above, the pulse speed and/or pulse intensity of the vibrations of the vibration actuators 40 may be varied as the ultrasound probe 14 is moved closer to appropriate position to image the target anatomy and/or the target scan plane to indicate proximity and/or speed of movement to the operator of the ultrasound probe 14.

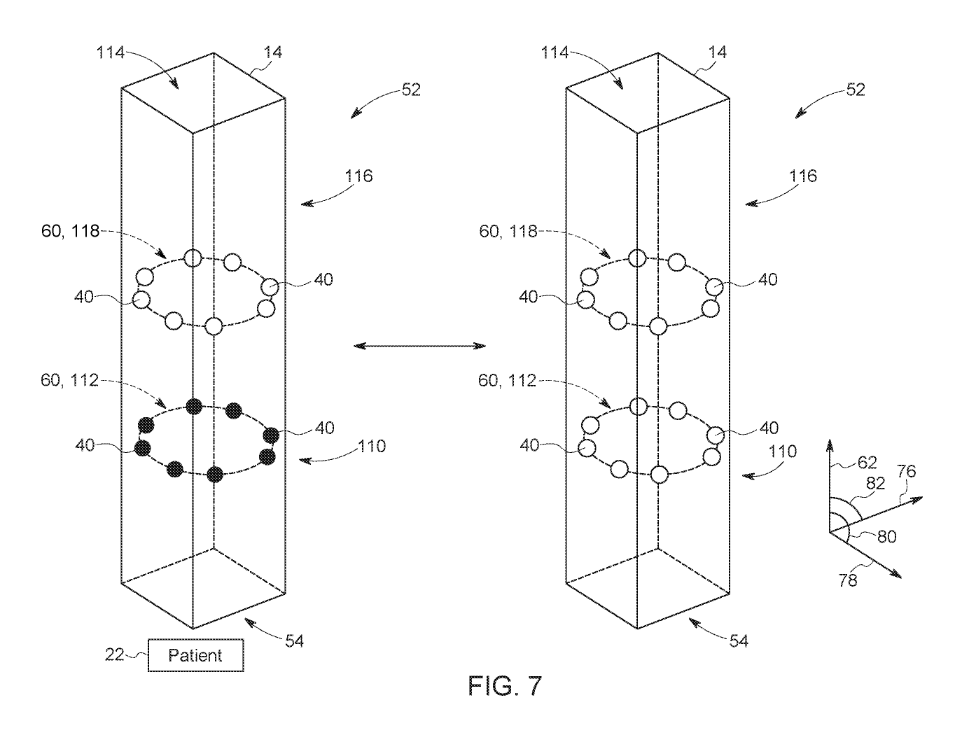

Similar vibration patterns may be used to guide compression (e.g., toward the tissue of the patient 22) and decompression (e.g., away from the tissue of the patient 22) movement of the ultrasound probe 14 along the axial axis 62 (e.g., Z-axis), as illustrated in FIG. 7. For example, the illustrated vibration pattern may be used to guide compression movement of the ultrasound probe 14 toward the tissue of the patient 22 and toward the patient-facing surface 54. The haptic guidance system 12, in some embodiments, may cause the vibration actuators 40 on a lower half 110 (e.g., half of the ultrasound probe 14 nearest the patient-facing surface 54) of the handle 52 of the ultrasound probe 14 to pulse, indicating to the operator of the ultrasound probe 14 to move the ultrasound probe 14 toward the tissue of the patient 22 in the direction of the pulses. The lower half 110 of the ultrasound probe 14 may include one or more of lower rings 112 (e.g., rings 60 nearest the patient-facing surface 54) of the rings 60 of the vibration actuators 40. In some embodiments, the pulse length, pulse repetition rate, and/or pulse intensity may vary, as discussed above with reference to FIG. 3, to indicate a distance from and/or to guide a speed of the movement of the ultrasound probe 14 to the appropriate position to sufficiently image the target anatomy in the target scan plane.

The opposite vibration pattern may be used to guide decompression movement of the ultrasound probe 14 in the opposite direction away from the tissue of the patient 22 and toward a non-patient-facing surface 114 (e.g., surface opposite the patient-facing surface). That is, the haptic guidance system 12 may cause the vibration actuators 40 on an upper half 116 (e.g., half of the ultrasound probe 14 opposite the patient-facing surface 54 and nearest the non-patient-facing surface 114) of the handle 52 of the ultrasound probe 14 to pulse, indicating to the operator of the ultrasound probe 14 to move the ultrasound probe 14 away from the tissue of the patient 22 in the direction of the pulses. The upper half 116 of the ultrasound probe 14 may include one or more of upper rings 118 (e.g., rings 60 nearest the non-patient-facing surface 114) of the rings 60 of the vibration actuators 40. In some embodiments, the pulse length, pulse repetition rate, and/or pulse intensity may vary, as discussed above, to indicate a distance from and/or to guide a speed of the movement of the ultrasound probe 14 to the appropriate position to sufficiently image the target anatomy in the target scan plane.

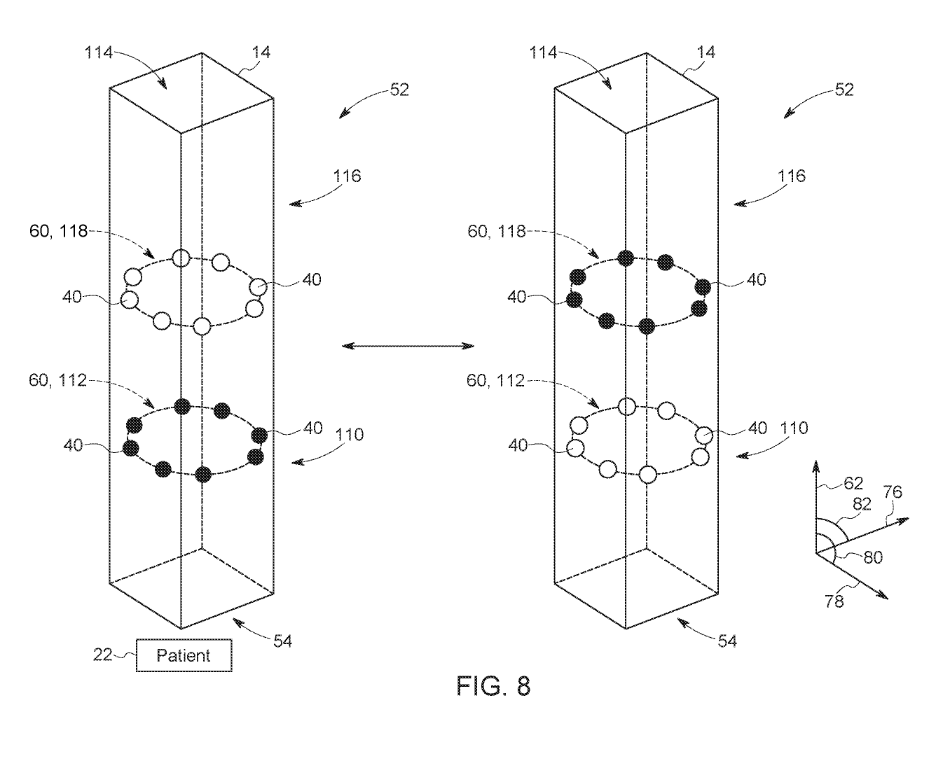

FIG. 8 illustrates an alternate vibration pattern that may be used to guide compression (e.g., toward the tissue of the patient 22) and decompression (e.g., away from the tissue of the patient 22) movement of the ultrasound probe 14 along the axial axis 62 (e.g., Z-axis). For example, the illustrated vibration pattern may be used to guide compression movement of the ultrasound probe 14 toward the tissue of the patient 22 and toward the patient-facing surface 54. The haptic guidance system 12, in some embodiments, may cause the vibration actuators 40 on the upper half 116 of the handle 52 of the ultrasound probe 14 opposite the patient-facing surface 54 of the ultrasound probe 14, such as the upper ring 118, to pulse in one or more short pulses, and then cause vibration actuators 40 on the lower half 110 of the handle 52 of the ultrasound probe 14 nearest the patient-facing surface 54 of the ultrasound probe 14, such as the lower ring 112, to pulse in one or more long pulses indicating movement in that direction toward the tissue of the patient 22. As such, the vibration pattern may include alternating short pulses on the upper half 116 of the ultrasound probe 14 and long pulses on the lower half 110 of the ultrasound probe 14 nearest the patient-facing surface 54 of the ultrasound probe 14. In some embodiments, the pulse length, pulse repetition rate, and/or pulse intensity may vary, as discussed above with reference to FIG. 3, to indicate a distance from and/or to guide a speed of the movement of the ultrasound probe 14 to the appropriate position to sufficiently image the target anatomy in the target scan plane. Additionally, as discussed above, the ultrasound probe 14 may include any number of rings 60. As such, the vibration pattern may be cycled through each ring 60. For example, if the ultrasound probe 14 includes 3 rings 60, the haptic guidance system 12 may cause the vibration actuators 40 on the upper half 116 of the handle 52 of the ultrasound probe 14 opposite the patient-facing surface 54 of the ultrasound probe 14, such as the upper ring 118, to pulse in one or more short pulses, then cause a middle ring 60 to pulse in one or more short pulses, and then cause vibration actuators 40 on the lower half 110 of the handle 52 of the ultrasound probe 14 nearest the patient-facing surface 54 of the ultrasound probe 14, such as the lower ring 112, to pulse in one or more long pulses indicating movement in that direction toward the tissue of the patient 22.

The opposite vibration pattern may be used to guide decompression movement of the ultrasound probe 14 in the opposite direction away from the tissue of the patient 22 and toward a non-patient-facing surface 114 (e.g., surface opposite the patient-facing surface). That is, the haptic guidance system 12, may cause the vibration actuators 40 on the lower half 110 of the handle 52 of the ultrasound probe 14 nearest the patient-facing surface 54 of the ultrasound probe 14, such as the lower ring 112, to pulse in one or more short pulses, and then cause vibration actuators 40 on the upper half 116 of the handle 52 of the ultrasound probe 14 opposite the patient-facing surface 54 of the ultrasound probe 14, such as the upper ring 118, to pulse in one or more long pulses indicating movement in that direction away from the tissue of the patient 22. As such the vibration pattern may include alternating short pulses on the lower half 110 of the ultrasound probe 14 and long pulses on the upper half 116 of the ultrasound probe 14 opposite the patient-facing surface 54 of the ultrasound probe 14. In some embodiments, the pulse length, pulse repetition rate, and/or pulse intensity may vary as discussed above to indicate a distance from and/or to guide a speed of the movement of the ultrasound probe 14 to the appropriate position to sufficiently image the target anatomy in the target scan plane.

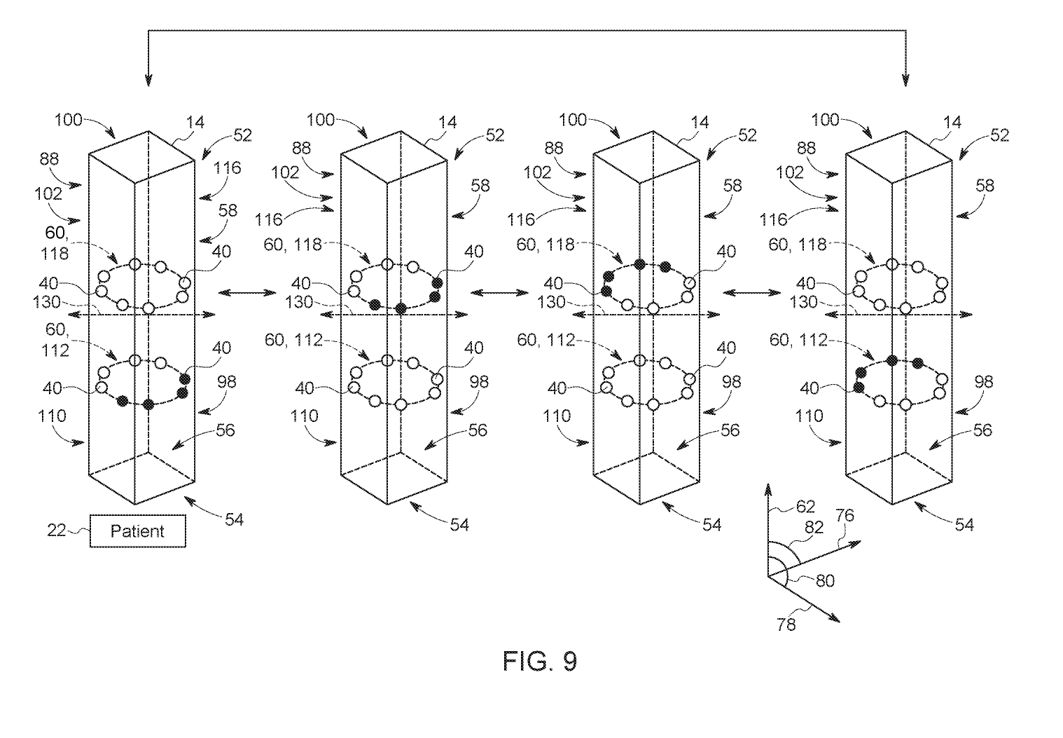

To guide orientation of the ultrasound probe 14 and the patient-facing surface 54 of the ultrasound probe 14, and thus the transducer array 18, within the three other degrees-of-freedom, circular vibration patterns may be used, which the operator may follow to adjust the ultrasound probe 14 into the desired orientation. For example, FIG. 9 illustrates an embodiment of a vibration pattern that may be used to guide tilting movement of the ultrasound probe 14 forward or backward along the elevation direction to increase or decrease the elevation angle 80. As such, the illustrated vibration pattern may be used guide tilting movement of the ultrasound probe 14 such that the front side 56 is moved closer to or farther from the patient 22, thus increasing or decreasing the elevation angle 80 between the axial axis 62 and the Y-axis 78 (e.g., between the front side 56 of the ultrasound probe 14 and the patient 22).

For example, the illustrated vibration pattern may be used to guide tilting movement of the handle 52 of the ultrasound probe 14 backward, thus increasing the elevation angle 80 between the patient 22 and the front side 56 of the ultrasound probe 14. The haptic guidance system 12, in some embodiments, may cause the vibration actuators 40 of the front half 98 of the lower ring 112 (e.g., the lower half 110) to pulse, then cause the vibration actuators 40 of the front half 98 of the upper ring 118 (e.g., the upper half 116) to pulse, then cause the back half 102 of the upper ring 118 to pulse, and then cause the back half 102 of the lower ring 112 to pulse, thus creating a circular vibration pattern. As such, the vibration pattern may move from the front side 56 nearest the patient-facing surface 54 of the ultrasound probe 14, to the front side 56 opposite the patient-facing surface 54 of the ultrasound probe 14, then to the back side 100 opposite the patient-facing surface 54 of the ultrasound probe 14, and then to the back side 100 nearest the patient-facing surface 54 of the ultrasound probe 14.

In this manner, the circular vibration pattern may rotate around an axis 130 of the ultrasound probe 14 extending from the second side 88 to the first side 58 of the ultrasound probe 14. This circular pattern may indicate to the operator to follow the circular pattern to tilt the handle 52 of the ultrasound probe 14 to increase the elevation angle 80 between the front side 56 of the ultrasound probe 14 and the patient 22, and thus cause the patient-facing surface 54 of the ultrasound probe 14 to tilt forward (e.g., tilt the patient-facing surface 54 away from the operator). In some embodiments, the pulse length, pulse repetition rate, and/or pulse intensity may vary, as discussed above with reference to FIG. 3, to indicate a distance from and/or to guide a speed of the movement of the ultrasound probe 14 to the appropriate orientation to sufficiently image the target anatomy in the target scan plane.

The opposite vibration pattern may be used to guide tilting movement of the handle 52 of the ultrasound probe 14 forward, thus decreasing the elevation angle 80 between the patient 22 and the front side 56 of the ultrasound probe 14. That is, the haptic guidance system 12 may cause the vibration actuators 40 of the front half 98 of the lower ring 112 to pulse, then cause the vibration actuators 40 of the back half 102 of the lower ring 112 to pulse, then cause the back half 102 of the upper ring 118 to pulse, and then cause the front half 98 of the upper ring 118 to pulse, thus creating a circular vibration pattern. As such, the vibration pattern may move from the front side 56 nearest the patient-facing surface 54 of the ultrasound probe 14, to the back side 100 nearest the patient-facing surface 54 of the ultrasound probe 14, then to the back side 100 opposite the patient-facing surface 54 of the ultrasound probe 14, and then to the front side 98 opposite the patient-facing surface 54 of the ultrasound probe 14. In this manner, the circular vibration pattern may rotate in the opposite direction around the axis 130 of the ultrasound probe 14 extending from the second side 88 to the first side 58 of the ultrasound probe 14. This circular pattern may indicate to the operator to follow the circular pattern to tilt the ultrasound probe 14 to decrease the elevation angle 80 between the front side 56 of the ultrasound probe 14 and the patient 22, and thus cause the patient-facing surface 54 of the ultrasound probe 14 to tilt backward (e.g., tilt the patient-facing surface toward the operator). In some embodiments, the pulse length, pulse repetition rate, and/or pulse intensity may vary, as discussed above, to indicate a distance from and/or to guide a speed of the movement of the ultrasound probe 14 to the appropriate orientation to sufficiently image the target anatomy in the target scan plane.

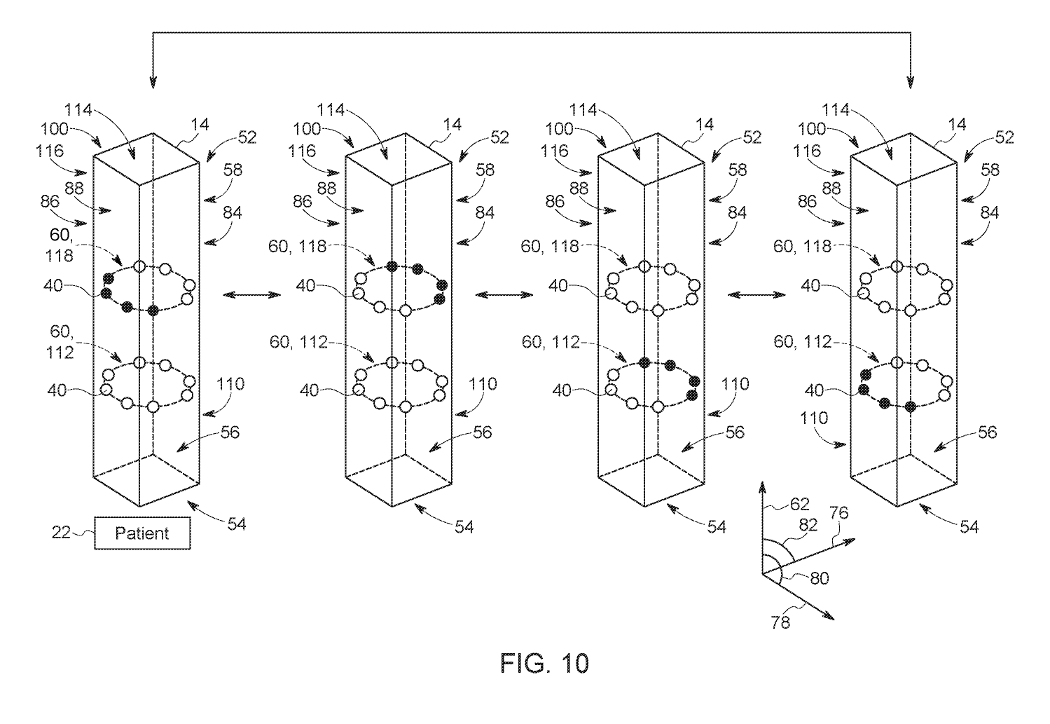

Similarly, FIG. 10 illustrates an embodiment of a vibration pattern that may be used to guide rocking movement of the ultrasound probe 14 left or right along the azimuth direction to increase or decrease the azimuth angle 82 (e.g., horizontal angle). For example, the illustrated vibration pattern may be used to guide rocking movement of the handle 52 of the ultrasound probe 14 toward the first direction (e.g., left) such that that first side 58 is moved closer to the tissue of the patient, thus decreasing the azimuth angle 82 between the axial axis 62 and the X-axis 76 (e.g., between the first side 58 of the ultrasound probe and the patient 22). The haptic guidance system 12, in some embodiments, may cause the vibration actuators 40 of the second half 86 of the upper ring 118 (e.g., the upper half 116) to pulse, then cause the vibration actuators 40 of the first half 84 of the upper ring 118 to pulse, then cause the first half 84 of the lower ring 112 (e.g., the lower half 110) to pulse, and then cause the second half 86 of the lower ring 112 to pulse, thus creating a circular vibration pattern. As such, the vibration pattern may move from the second side 88 (e.g., right side) opposite the patient-facing surface 54 of the ultrasound probe 14, to the first side 58 opposite the patient-facing surface 54 of the ultrasound probe 14, then to the first side 58 nearest the patient-facing surface 54 of the ultrasound probe 14, and then to the second side 88 nearest the patient-facing surface 54 of the ultrasound probe 14.

In this manner, the circular vibration pattern may rotate around the axial axis 62 of the ultrasound probe 14 extending from patient-facing surface 54 to the non-patient-facing surface 114 of the ultrasound probe 14. This circular pattern may indicate to the operator to rock the handle 52 of the ultrasound probe 14 toward the first direction to decrease the azimuth angle 82 between the first side 58 of the ultrasound probe 14 and the patient 22, and thus cause the patient-facing surface 54 of the ultrasound probe to face more toward the second direction. In some embodiments, the pulse length, pulse repetition rate, and/or pulse intensity may vary, as discussed above with reference to FIG. 3, to indicate a distance from and/or to guide a speed of the movement of the ultrasound probe 14 to the appropriate orientation to sufficiently image the target anatomy in the target scan plane.

The opposite vibration pattern may be used to guide rocking movement of the handle 52 the ultrasound probe 14 in the opposite direction toward the second direction (e.g. right), thus increasing the azimuth angle 82 between the first side 58 of the ultrasound probe 14 and the patient 22 and decreasing the azimuth angle 82 between the second side 88 of the ultrasound probe 14 and the patient 22. That is, the haptic guidance system 12 may cause the vibration actuators 40 of the second half 86 of the upper ring 118 to pulse, then cause the vibration actuators 40 of the second half 86 of the lower ring 112 to pulse, then cause the first half 84 of the lower ring 112 to pulse, and then cause the first half 84 of the upper ring 118 to pulse, thus creating a circular vibration pattern. As such, the vibration pattern may move from the second side 88 opposite the patient-facing surface 54 of the ultrasound probe 14, to the second side 88 nearest the patient-facing surface 54 of the ultrasound probe 14, then to the first side 58 nearest the patient-facing surface 54 of the ultrasound probe 14, and then to the first side 58 nearest the patient-facing surface 54 of the ultrasound probe 14. In this manner, the circular vibration pattern may rotate around an axis 132 of the ultrasound probe 14 extending from the front side 56 to the back side 100 of the ultrasound probe 14. As discussed above, the pulse speed and/or intensity may be varied as the ultrasound probe is moved closer to the desired position to indicate proximity and/or speed of movement to the operator. In some embodiments, the pulse length, pulse repetition rate, and/or pulse intensity may vary, as discussed above, to indicate a distance from and/or to guide a speed of the movement of the ultrasound probe 14 to the appropriate orientation to sufficiently image the target anatomy in the target scan plane.

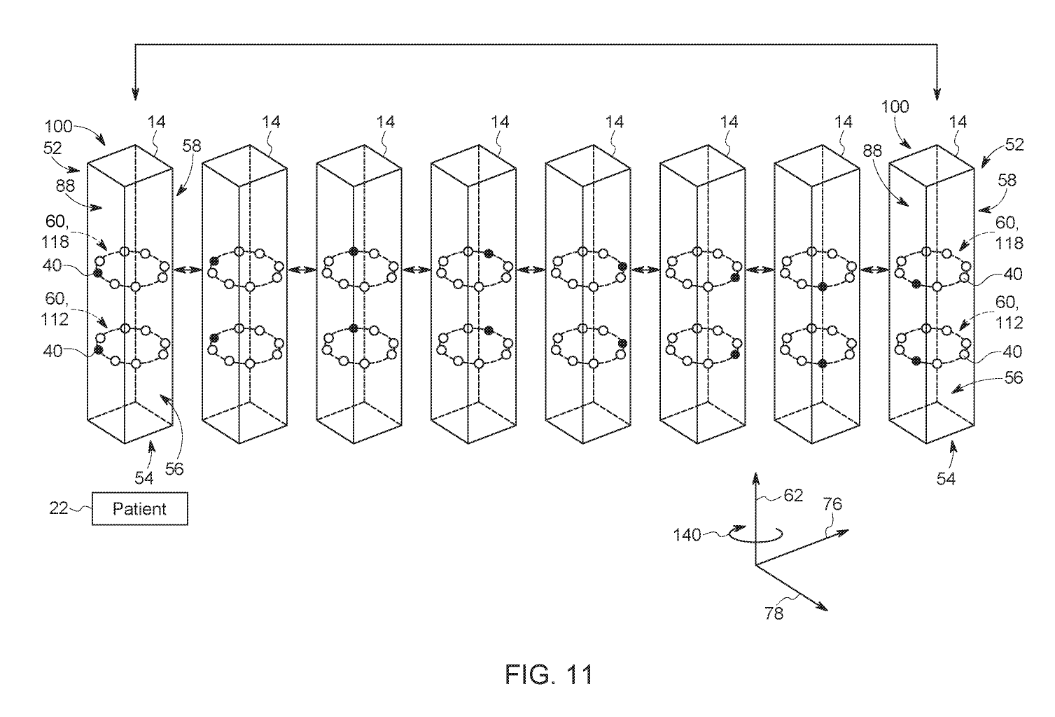

To cause a rotation motion of the ultrasound probe 14 in a circumferential direction 140 around the axial axis 62 (e.g., Z-axis) in a clockwise or counterclockwise direction, the haptic guidance system 12 may cause the vibration actuators 40 to vibrate in a circular pattern, as illustrated in FIG. 11. The haptic guidance system 12, in some embodiments, may cause the axially aligned vibration actuators 40 of each of the rings 60 of vibration actuators 40 to pulse in order around the axial axis 62 of the ultrasound probe 14. The operator may follow the pattern of the vibration to rotate the ultrasound probe clockwise or counterclockwise about the axial axis 62. To cause the operator to rotate the ultrasound probe 14, and thus the patient-facing surface 54 of the ultrasound probe 14, clockwise, as in the illustrated embodiment, the vibration pattern may move from the front side 56 of the ultrasound probe 14, to the second side 88 of the ultrasound probe 14, to the back side 100 of the ultrasound probe 14, to the first side 58 of the ultrasound probe 14. In this manner, the circular vibration pattern may rotate around the axial axis 62 (e.g., Z-axis) of the ultrasound probe 14. This circular pattern may indicate to the operator to rotate the ultrasound probe 14 clockwise about the axial axis 62. The opposite vibration pattern, may be used to guide rotation movement of the ultrasound probe 14 counterclockwise about the axial axis 62. In some embodiments, the pulse length, pulse repetition rate, and/or pulse intensity may vary, as discussed above with reference to FIG. 3, to indicate a distance from and/or to guide a speed of the movement of the ultrasound probe 14 to the appropriate orientation to sufficiently image the target anatomy in the target scan plane.

The examples discussed above of vibration patterns that may be used to guide the operator to position and orient the ultrasound probe in the appropriate position and/or orientation to capture the target scan plane of the target anatomy. The examples discussed of the vibration patterns discussed above are not intended to be limiting, but only as examples of possible vibration, or excitation, patterns. Other vibration patterns may encode or indicate suggested motion, and may be used to guide movement of the ultrasound probe 14. For example, the example vibration patterns discussed above are implemented spatially on the ultrasound probe 14, such that the vibration patterns approximately align with the natural spatial locations (e.g., left, right, up, down, forward, backward, rotate). However, in some embodiments, motion direction information may be indicated directly in the vibration patterns with less emphasis on the spatial orientation of the vibration actuators 40. That is, in some embodiments, all of the vibration actuators 40 of the ultrasound probe 14 or a portion of the vibration actuators 40 may vibrate in a particular pattern to indicate movement in a particular direction or degree-of-freedom. For example, in one embodiment, all of the vibration actuators 40 of the ultrasound probe 14 may vibrate in a short-short-long-rest pattern and then repeat to indicate suggested compression movement toward the patient 22. Additionally, all of the vibration actuators 40 of the ultrasound probe 14 may vibrate in a long-short-short-rest pattern and then repeat to indicate suggested decompression movement away from the patient 22. Such vibration patterns that do not rely on the spatial orientation of the vibration actuators 40 and variations of such vibration patterns maybe extrapolated to indicate motion in all six degrees-of-freedom. Additionally or alternatively, vibration patterns that do not rely on the spatial orientation of the vibration actuators 40 may be used in combination with the vibration patterns described in FIGS. 3-11 to convey suggested movement of the ultrasound probe simultaneously in multiple guidance methods to guide the operator to move the probe to the appropriate position and orientation to image the target anatomy in the target scan plane, as different operators may be more receptive to different vibration pattern types.

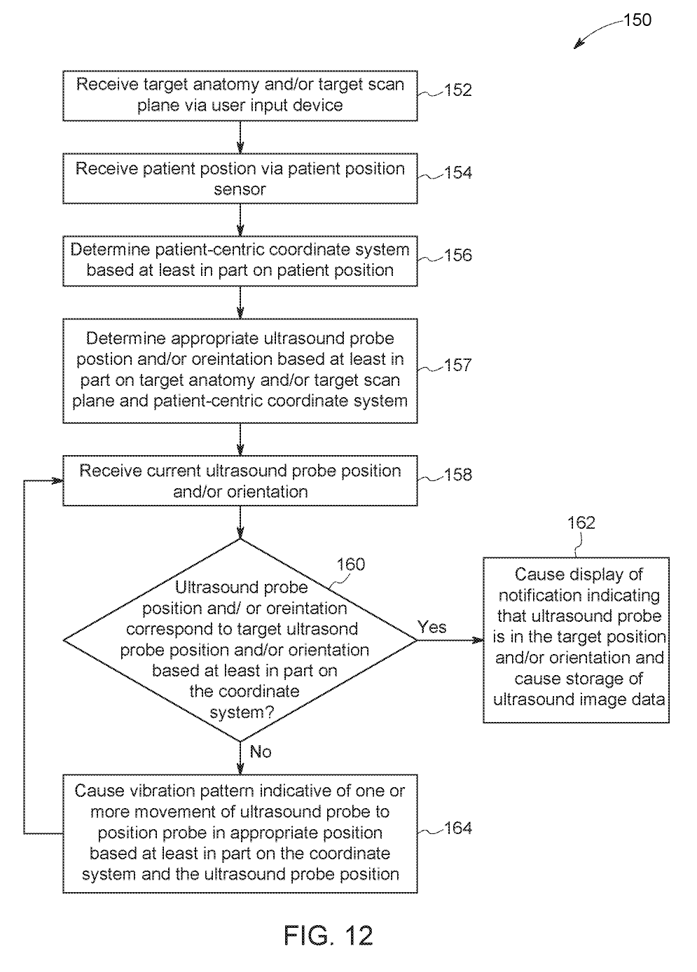

FIG. 12 illustrates an embodiment of a method 150 for guiding movement of the ultrasound probe using the haptic guidance system to position the ultrasound probe 14 in the appropriate position and/or orientation to sufficiently image the target anatomy in the target scan plane. At step 152, the controller 24 of the haptic guidance system 12 may receive the target anatomy and/or the target scan plane of the target anatomy to be imaged via the user input device. At step 154, the controller 24 may receive a position of the patient 22 via the patient position sensor 33. As such, the controller 24 may receive one or more signals from the patient position sensor 33 indicative of the position of the patient 32 about the imaging space 35. Additionally or alternatively, in some embodiments, the position of the patient may be determined based on the live ultrasound image data from the ultrasound probe 14. At step 156, the controller 24 may determine the patient-centric coordinate system based at least in part on the received patient position. In some embodiments, the patient-centric coordinate system for the current patient 22 may be saved in the memory 32, and in some embodiments, this saved coordinate system may be updated based on the current position of the patient 22. At step 157, the controller 24 may determine the appropriate position and/or orientation of the ultrasound probe 14 based at least in part on the target anatomy, the target scan plane, the patient-centric coordinate system, and/or the live ultrasound image data.

At step 158, the controller 24 may receive a current position and or orientation of the ultrasound probe 14 from the probe position sensor 36. At step 160, the controller 24 may determine whether the position and/or orientation of the ultrasound probe 14 corresponds to the appropriate position to image the target anatomy in the target scan plane based at least in part on the patient-centric coordinate system and the position of the ultrasound probe 14. In some embodiments, the controller 24 may use the live ultrasound image data captured by the ultrasound probe 14 as feedback to determine patient position, the appropriate position and orientation of the ultrasound probe 14, and/or if the ultrasound probe 14 is in the appropriate position and/or orientation. The target anatomy in the time series of images may be recognized and the corresponding ultrasound probe positions during that time series are used to derive a target probe position. If the controller 24 determines that the ultrasound probe 14 is in the appropriate position and/or orientation to sufficiently image the target anatomy in the target scan plane, the method 150 may continue to step 162 in which the controller 24 may cause display of a notification, such as a visual or audible notification, and/or the controller 24 may send a signal to the vibration actuators 40 of the ultrasound probe 14 indicative of a particular pulse pattern indicating to the operator of the ultrasound probe 14 that the ultrasound probe 14 is in the appropriate position and/or orientation. Additionally or alternatively, in some embodiments, the controller 24 may cause automatic storage in the memory 32 of the live ultrasound image data once it is determined that the ultrasound probe is in the appropriate position and/or orientation. If at step 160 the controller 24 determines that the ultrasound probe is not in the appropriate position and/or orientation to sufficiently image the target anatomy in the target scan plane, the controller 24 may cause one or more vibration pattern indicative of one or more movements of the ultrasound probe 14 to position the ultrasound probe in the appropriate position based at least in part on the patient-centric coordinate system, the ultrasound probe 14 position, and/or the ultrasound image, at step 164.

The vibration pattern at step 164 may indicate to the operator of the ultrasound probe 14 to move the ultrasound in a particular direction or manner to achieve the appropriate position and/or orientation to sufficiently image the target anatomy in the target scan plane. The controller 24 may then continue to repeat steps 158, 160, and 164 until the controller 24 determines that the ultrasound probe 14 is in the appropriate position and/or orientation to sufficiently image the target anatomy in the target scan plane.

The haptic guidance system disclosed provides positioning guidance of the ultrasound probe by utilizing an alternate sense perception of the operator (e.g., tactile sense perception). Such haptic feedback may provide more intuitive guidance to the operator instead of visual cues, or voice guidance, as the operator is already focused on viewing the ultrasound machine and communicating with the patient. Additionally, an array of vibration actuators may allow guidance of the ultrasound probe in all six degrees-of-freedom and may accommodate multiple grip styles. Further, in the big picture, an operator-independent ultrasound imaging system, such as the system described above including the haptic guidance system, may allow novice users to successfully image the target anatomy in the target scan plane. Thus, ultrasound technology may become a more common technology used in every physician's office.

While only certain features of the present disclosure have been illustrated and described herein, many modifications and changes will occur to those skilled in the art. It is, therefore, to be understood that the appended embodiments are intended to cover all such modifications and changes as fall within the scope of the disclosure.

1. An ultrasound imaging system, comprising:
an ultrasound probe comprising a handle comprising a plurality of haptic actuators; and
a monitor comprising a memory and a processor, wherein the processor is communicatively coupled to the ultrasound probe and configured to:
determine a current position of the ultrasound probe relative to the target region of interest;
determine whether the current position of the ultrasound probe corresponds to a target position of the ultrasound probe relative to a target region of interest of a patient, wherein the target region of interest comprises a target anatomy and a target scan plane; and
output, in response to determining that the current position of the ultrasound probe relative to the target region of interest does not correspond to the target position of the ultrasound probe, a control signal to one or more of the haptic actuators of the plurality of haptic actuators, wherein the control signal causes one or more of the haptic actuators to activate in an activation pattern indicative of a suggested movement of the ultrasound probe to position the ultrasound probe in the target position.

2. The ultrasound imaging system of claim 1, wherein the processor is further configured to receive a patient position via a patient position sensor of the ultrasound imaging system and to determine a coordinate system based at least in part on the patient position.

3. The ultrasound imaging system of claim 2, wherein the processor is configured to determine whether the current position of the ultrasound probe corresponds to the target position based at least in part on the determined coordinate system.

4. The ultrasound imaging system of claim 2, wherein the patient position sensor comprises one or more of cameras, contact sensors, weight sensors, an ultrasound probe comprising integrated position tracking, ultrasound image data from the ultrasound probe, or any combination thereof.

5. The ultrasound imaging system of claim 1, wherein the haptic actuators comprise vibration motors.

6. The ultrasound imaging system of claim 1, wherein the haptic actuators comprise motion actuators, electrodes, or deformable fluid filled chambers.

7. The ultrasound imaging system of claim 1, wherein the target position of the ultrasound probe comprises a target position and a target orientation of the ultrasound probe relative to the region of interest of the patient.

8. The ultrasound imaging system of claim 1, wherein the activation pattern is indicative of one or more suggested movements in six degrees-of-freedom.

9. An ultrasound probe, comprising:
a patient-facing surface comprising one or more transducers; and
a handle comprising a plurality of haptic actuators, wherein the haptic actuators are configured to actuate in a plurality of actuation patterns to indicate a suggested movement of the handle in six degrees-of-freedom, and wherein each actuation pattern of the plurality of actuation patterns modulates in intensity, speed, or both as the patient-facing surface of the ultrasound probe is moved a distance from a target position relative to a patient.

10. The ultrasound probe of claim 9, comprising a probe position sensor configured to measure a position of the ultrasound probe relative to the patient.

11. The ultrasound probe of claim 9, wherein the haptic actuators are disposed below a housing surface of the ultrasound probe and are arranged in two or more actuation rings axially offset from each other about an axial axis of the ultrasound probe.

12. The ultrasound probe of claim 11, wherein each actuation ring comprises one or more of the haptic actuators per side of the ultrasound probe.

13. The ultrasound probe of claim 9, wherein the actuation patterns communicate suggested movement of the ultrasound probe relative to an X-axis, a Y-axis, and a Z-axis to position the ultrasound probe in the target position relative to the patient, wherein suggested movement relative to the Z-axis comprises suggested compression movement and decompression movement of the ultrasound probe relative to the patient, and wherein compression movement comprises movement of the ultrasound probe toward the patient.

14. The ultrasound probe of claim 9, wherein the actuation patterns communicate suggested movement of the ultrasound probe relative to an elevation angle and an azimuth angle to orient the patient-facing surface of the ultrasound probe relative to the patient.

15. The ultrasound probe of claim 9, wherein the actuation patterns communicate suggested rotation movement of the ultrasound probe circumferentially about an axial axis of the ultrasound probe.

16. The ultrasound probe of claim 9, wherein each haptic actuator of the plurality of haptic actuators comprises a shell configured to spatially contain the respective haptic actuator such that actuation of the respective haptic actuator in the plurality of actuation patterns is location specific.

17. A method, comprising:
receiving, via a processor, a target region of interest of a patient to be imaged via an ultrasound probe of an ultrasound imaging system and a target scan plane of the target region of interest;
determining, via the processor, a target imaging position of the ultrasound probe based at least in part on the target region of interest and the target scan plane;
determining, via the processor, a current position of the ultrasound probe relative to the target region of interest, the desired scan plane, or both;
determining, via the processor, whether the current position of the ultrasound probe corresponds to the target imaging position of the ultrasound probe; and
outputting, via the processor, in response to determining that the current position of the ultrasound probe relative to the target region of interest, the target scan plane, or both, does not correspond to the target position of the ultrasound, a control signal to a plurality of haptic actuators disposed on a handle of the ultrasound probe, wherein the control signal causes one or more of the haptic actuators to actuate in an actuation pattern indicative of a suggested movement of the ultrasound probe.

18. The method of claim 17, comprising:
determining, via the processor, a position of the patient relative to the ultrasound imaging system; and
determining, via the processor, a patient-centric coordinate system based at least in part on the position of the patient.

19. The method of claim 18, wherein determining whether the current position of the ultrasound probe corresponds to the target imaging position of the ultrasound probe is based at least in part on the patient-centric coordinate system.

20. The method of claim 17, wherein the haptic actuators comprise vibration actuators, and, wherein the actuation pattern comprises a plurality of actuation patterns each indicative of a suggested movement of the ultrasound probe in six degrees-of-freedom, and wherein the plurality of actuation patterns modulate in intensity, speed, or both as the ultrasound probe is moved a distance from the target imaging position.
